(19) United States
(12) Patent Application Publication
(10) Pub. No.: US 2015/0158904 A1
(43) Pub. Date: Jun. 11, 2015

(54) SYNTHESIS OF TANTALUM SOL-GEL AND PRODUCTION METHOD OF MICROEXTRACTION SYRINGE FOR THE PURPOSE OF ENRICHMENT OF PHOSPHOPEPTIDES

(71) Applicants: Bekir Salih, ANKARA (TR); Omur Celikbicak, ANKARA (TR); Mehmet Atakay, ANKARA (TR)

(72) Inventors: Bekir Salih, ANKARA (TR); Omur Celikbicak, ANKARA (TR); Mehmet Atakay, ANKARA (TR)

(73) Assignee: BEKIR SALIH, ANKARA (TR)

(21) Appl. No.: 14/261,855

(22) Filed: Apr. 25, 2014

(30) Foreign Application Priority Data
Dec. 5, 2013 (TR) 2013/14066

(51) Int. Cl. C07K 1/18; B01J 41/10; B01J 41/02

(52) U.S. Cl. CPC C07K 1/18; B01J 41/02; B01J 41/10

(57) ABSTRACT

The present invention relates to a production method of sol-gel (100) comprising tantalum metal and a method (200) concerning use of the sol-gel produced for enrichment of phosphopeptides by means of a microextraction syringe. The syringe which is filled with the sol-gel comprising tantalum metal enables to make analysis of natural samples, that comprise low amount of phosphoprotein, with high precision and in a very short time and it shows compliance with mass spectrometric techniques. Thus, it can be used for application of fast and practical analytical methods for the analysis made in phosphoproteomics studies wherein mass spectrometric techniques are used.

### CROSS REFERENCE TO THE RELATED APPLICATION

This application claims the benefit to Turkish Patent Application No 2013/14066 filed Dec. 3, 2013, the content of which is incorporated herein by reference.

### TECHNICAL FIELD

The present invention relates to a production method of sol-gel comprising tantalum metal and a method concerning use of the sol-gel produced for enrichment of phosphopeptides by means of a microextraction syringe.

### BACKGROUND OF THE INVENTION

In order to increase enrichment of phosphopeptides and their assay sensitivities, various techniques are developed with each passing day. Methods based on covalent bonding or non-covalent interactions are used with the purpose of enriching phosphopeptides. However, use of enrichment methods based on non-covalent interactions is more common. In order to analyse phosphopeptides, which are included in a natural sample, by means of mass spectrometric techniques with high selectivity and sensitivity it is aimed to develop practical phosphopeptide enrichment methods wherein reusable and economical materials are used. By means of using these methods and materials, it is contributed to medical applications aiming to create diagnosis and treatment methods of diseases such as cancer or Alzheimer's disease occurring as a result of basic cellular activity disorders.

In commonly used phosphopeptide enrichment methods, principle of interactions of phosphate groups on peptides with their metal oxides is utilized. Purification studies can be done with chromatographic methods by taking advantage of negatively-charged phosphate groups included in structures of phosphopeptides. Particularly, affinity chromatography methods based on principle of interactions of negatively-charged phosphate groups with their metal cations is frequently used. There are many different chromatographic methods wherein metals, developed with this approach, such as iron (LI-YUAN, 2010), gallium (SALIM, 2012) or metal oxides such as titanium dioxide (KRENKOVA 2013), zirconium dioxide (NELSON, 2010) are used

Materials such as titanium dioxide (TiO₂) and zirconium dioxide (ZrO₂) also become suitable for binding of phosphopeptides at low pH values. By means of the studies done it is shown that binding affinity of ZrO₂ to phosphate group is a lot more than affinity thereof to carboxylate anions (BLACKWELL, 1991). In addition, ZrO₂ has been used as a chromatographic material for a long time because of its physical and thermal stability. Using ZrO₂ surfaces having these features is suitable for studies of phosphopeptide enrichment. Although TiO₂ is the most commonly used materials of phosphopeptide enrichment on metal oxide surface, oxides of other various materials other than titanium are also used in studies of phosphopeptide enrichment. Fe₃O₄-coated AI₂O₃ (LI, 2007) or Ga₂O₃ (LI, 2008) magnetic microspheres were used in studies of phosphopeptide enrichment and successful results were obtained. Also, it was shown that SnO₂ (STURM, 2008) and Nb₂O₅ (FICARRO, 2008) metal oxides can be used in phosphopeptide enrichment.

Sol-gel materials attract attention due to the fact that they can be produced as having pre determinable pore size and shape; and because of their potentials in separations, adsorption, catalysis, drug release and chemical sensor applications needed in molecular identification. it cannot be said that amorphous metal oxide sol-gel materials are not as successful as acrylic-based pressed polymers in chemical applications. However, sol-gel materials have many advantages in comparison to organic polymers. For example, thermal stability of organic polymers is as low as it cannot be compared to sol-gels. In sol-gels, control of thickness, porosity and surface area is easy; selectivity and diffusion is also better than acrylic-based polymers (MARX, 2001; LAHAV, 2001).

Phosphoproteom analysis made with mass spectrometric methods are usually performed at two steps. The phosphoprotein to be analyzed is enzymatically disintegrated at first step whereas peptide units occurring as a result of enzymatic digestion are analyzed at the second step. Although this method appears to be useful, it may be confronted with some problems in applications wherein this approach is used. The fact that signals belonging to all of phosphopeptides in the medium in the peptide mass maps obtained cannot be observed, ion formation decreases as a result of the phosphate group increasing the acidity, other peptide ions in the medium suppress signals belonging to phosphopeptides, phosphopeptides with hydrophilic feature cannot usually hold on to column materials (reverse phase) used for purification of peptides sufficiently can be given as an example to these problems.

In order to overcome the difficulties encountered in phosphoprotein and phosphopeptide analysis which are made with mass spectrometric methods, it is aimed to develop selective enrichment methods. Enrichment process enables to make phosphopeptide analysis by eliminating the problem of ion suppression arising from existence of peptides not including phosphate group.

There is no enrichment or identification method which completely shows compliance for the whole of phosphoproteomics studies. In studies done at the present time, enrichment and purification methods of phosphoproteins or phosphopeptides must be selected depending on type of the sample to be analyzed. In fact currently available methods are successful at enrichment studies of a part of phosphoproteins in their way. It is considered that development of different and new methods showing compliance for each sample in this field will provide quite substantial benefit for proteomics studies.

An increase can be observed in amount of impurity in the enrichment medium depending on separation of metal ions from the material wherein they are included, in studies where materials including the metal ions are present with the purpose of phosphopeptide enrichment. In such a case, efficiency of purification considerably decreases due to the fact that the material used for enrichment is not stable.

Acidic peptides, which are included in the medium apart from phosphopeptides, may show interest to surfaces of metal oxides frequently used in studies of phosphopeptide enrichment such as TiO₂. Extra methods and Chemical additives may be used with the purpose of removing other acidic peptides and increasing selectivity of the enrichment method. Separation principles of anion or cation exchange materials used in enrichment of phosphopeptide are based on electrostatic interactions between types. Electrostatic interactions are random interactions selectivity of which is quite low. Even if types having opposite electrical charge are random they may tend to get together with such interactions. It is quite difficult to purify only small amount of phosphopeptides from among quite complex samples such as cell lysate, using such materials. Because of these reasons; durable, inexpensive, reusable phosphopeptide with high-selectivity can be alternative to enrichment methods wherein such materials are used.

The International Patent Document No. WO2012079549 A3 discloses that materials which can be used for the purpose of enrichment of phosphopeptide by performing surface modification with organometallic compounds comprising elements of 4B group such as zirconium, hafnium, titanium are developed.

The United States Patent Document No. U.S.20100125128 A1 presents ceramic hydroxyapatite material and analytical method which can be used in phosphopeptide enrichment applications. It is stated that phosphopeptides having different features can be separate from each other besides enrichment of phosphopeptide by means of this method.

The International Patent Document No. WO2010045710 A1 states that phosphorylated biomolecules such as phosphopeptides and phosphoproteins can be purified from the medium wherein they exist using an affinity matrix with metal ion content.

The United States Patent Document No. U.S.8003340 B2 proposes a purification process which is carried out based on the principle of precipitation of metal-phosphoprotein complexes obtained as a result of binding calcium, barium, molybdenum, cobalt ions to phosphoproteins selectively.

### SUMMARY OF THE INVENTION

An objective of the present invention is to realize a production method of sol-gel material which enables to make analysis of natural samples, that comprise phosphoproteins or phosphopeptides low concentration, of a syringe filled with sol-gel comprising a tantalum metal with high precision and in a very short time and works in line with mass spectrometric techniques; and a method for use of sol-gel material in enrichment of phosphopeptide.

Another objective of the present invention is to realize a production method of sol-gel material wherein the solid-phase microextraction material, that is developed using the sol-gel synthesized, is reusable; and a method for use of sol-gel material in enrichment of phosphopeptide.

A further objective of the present invention is to realize a production method of sol-gel material wherein a sol-gel with a content of tantalum metal having chemical stability, quite durable physically and suitable for use in natural sample analysis is produced; and a method for use of sol-gel material in enrichment of phosphopeptide.

A yet further objective of the present invention is to realize a production method of sol-gel material wherein the sol-gel with the content of tantalum metal synthesized is used without being subjected to calcination process at high temperature; and a method for use of sol-gel material in enrichment of phosphopeptide.

A production method of sol-gel and a method for use of sol-gel in enrichment of phosphopeptide realized to fulfil the objectives of the present invention are shown in the figures attached.

### DESCRIPTION OF THE DRAWINGS

FIG. 1 is a view of the flow chart concerning the inventive production method of sol-gel.

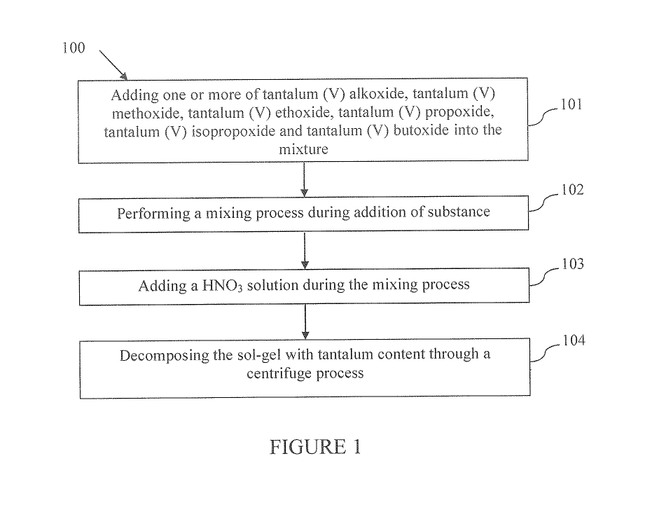

FIG. 2 is a view of the flow chart of the method for use of the inventive sol-gel in enrichment of phosphopeptide.

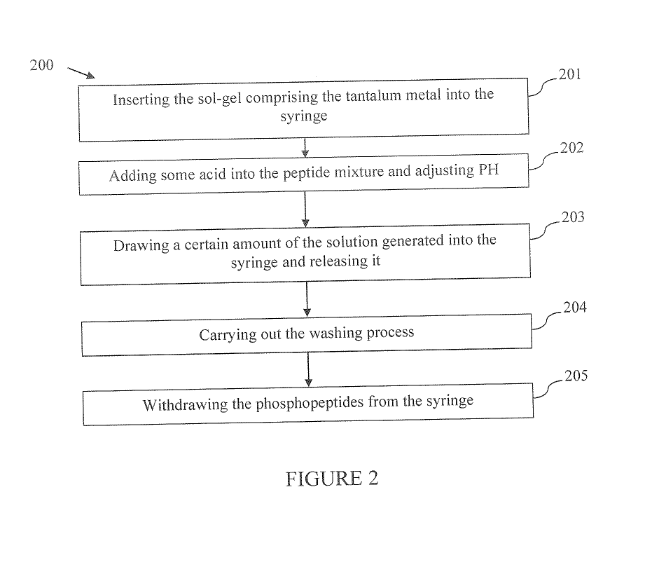

FIG. 3 is a mass spectrum of a milk sample form before (A) and after (B) the process of phosphopeptide enrichment is applied, which is generated as a result of analysis thereof with MALDI-MS (Matrix-assisted Laser Desorption/Ionization-Mass Spectrometry).

FIG. 4 is a view of the sol-gel network structure comprising tantalum.

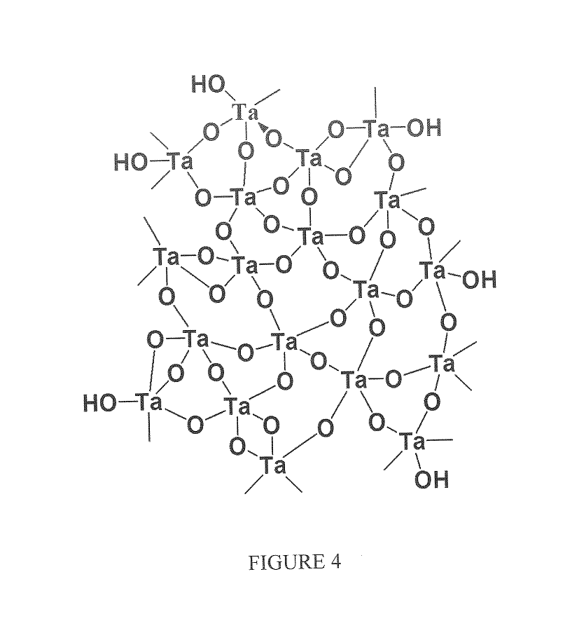

### DETAILED DESCRIPTION OF THE INVENTION

The inventive production method of sol-gel (100) comprises steps of:
adding one or more of tantalum (V) alkoxide, tantalum (V) methoxide, tantalum (V) ethoxide, tantalum (V) propoxide, tantalum (V) isopropoxide and tantalum (V) butoxide into the mixture (101);
performing a mixing process during addition of material (102);
adding an acid solution such as HNO₃, HCl, H₂SO₄, CH₃COOH, CF₃COOH etc. during the mixing process (103);
decomposing the sol-gel with tantalum content through a centrifuge process (104).

In the inventive method (100) before the addition of tantalum metal into the mixture (101), the solution comprising polyethylene glycol (PEG) (Mr-600-3000) in a ratio of 30-70% by volume is diluted with ethanol: water mixture (18:0.5-18:3.0, v/v) (1%, v/v). After the dilution process, one or more of tantalum (V) alkoxide, tantalum (V) methoxide, tantalum (V) ethoxide, tantalum (V) propoxide, tantalum (V) isopropoxide and tantalum (V) butoxide is/are added into the mixture (101). During addition of starting material, mixing process is carried on (102) quickly. While the mixing process continues, up to 0.1-0.5% of a concentrated acid solution such as HNO₃, HCl, H₂SO₄, CH₃COOH, CF₃COOH, etc. is added into the synthesis medium (103). Upon addition of acid during this process carried out at room temperature, it is observed that sol-gel particles with tantalum content occur (104).

In a preferred embodiment of the invention, the sol-gel with tantalum content obtained is washed and then taken into its final form after being dried. The sol-gel with tantalum content is washed with solvents such as ethanol, water and methanol in the preferred embodiment. Drying process is carried out at temperature of 40° C. in a vacuum environment between 2-200 mbars.

Whereas the method of phosphopeptide enrichment (200) which is realized using the sol-gel obtained comprises steps of:
inserting the sol-gel comprising the tantalum metal into the syringe (201);
adding some acid into the peptide mixture and adjusting PH (202);
drawing a certain amount of the solution generated into the syringe and releasing it (203);
carrying out the washing process (204); and
withdrawing the phosphopeptides from the syringe (205).

In the inventive enrichment method (200), after the sot-gel comprising the tantalum metal is inserted the into the syringe (201) firstly some mixture of acetonitrile:water:trifluoroacetic acid (1:1:0.005-0.3, v/v/v) is added into the peptide mixture Obtained as a result of enzymatic digestion and the PH is adjusted (202). PH value of the acid mixture is approximately between 1.0-3.0. In a preferred embodiment, 50 μL of the solution generated is drawn into the syringe and released (203). The said process is repeated for 2-6 times. And in order to remove impurities and peptides except phosphopeptides, the mixture of acetonitrile:water:trifluoroacetic acid (1:11:0.005-0.3, v/v/v) is drawn into the syringe and released. The said is also repeated for 2-6 times. The solution of 10-100 mM ammonium bicarbonate comprising ammonia in the washing process is pulled through the syringe (204) lastly and the phosphopeptides are withdrawn from the syringe (205).

The microextraction syringe which is filled with the sol-gel comprising tantalum metal enables to make analysis of natural samples, that comprise low amount of phosphoprotein, with high precision and in a very short time and it shows compliance with mass spectrometric techniques. Thus, it can be used for application of fast and practical analytical methods for the analysis made in phosphoproteomics studies wherein mass spectrometric techniques are used. The sot-gel synthesized has physical and chemical properties which will show compliance to ambient conditions in terms of being usable in methods of phosphopeptide enrichment. The solid-phase microextraction material which is developed using the sol-gel synthesized with the trials made is reusable. The sol-gel comprising tantalum metal has chemical stability, is quite durable physically and suitable for use in natural sample analysis.

The sol-gel comprising the tantalum metal synthesized can he used without being subjected to calcination process, which is defined as digestion of carbonates and hydrates with temperature effectin order to obtain oxide components, at high temperature. The process of phosphopeptide enrichment is realized over interactions of phosphopeptides with positive metal centers on the sol-gel surface comprising tantalum of the electron-rich phosphate groups. Therefore, phosphopeptides are expected to be negatively charged and the tantalum centers on the surface are expected to be positively charged during the interaction between the said types. Electron need of metal on the surface is provided by the oxygen atoms due to the more regular structure which is composed as a result of the oxidation occurring by calcination of the sol-gel surface at high temperature. Therefore, phosphopeptides' interests for sol-gel surface decrease. However, it is considered that using sol-gel after steps of synthesis and washing steps required will increase efficiency of the process of phosphopeptide enrichment. The sol-gel which comprises tantalum metal used without applying any calcination process, enables to obtain quite successful results at the step of phosphopeptide enrichment.

With the purpose of testing efficiency of the sol-gel obtained in the process of phosphopeptide enrichment, besides many mixtures cow's milk showing effect of different natural sample and high matrix is used. Proteins involved in milk are denaturated before the enrichment process and broken into peptide fragments by the trypsin enzyme. As a result of this process, it is ensured that phosphopeptides are separated selectively using the complex peptide mixture obtained and the syringe developed from within matrix.

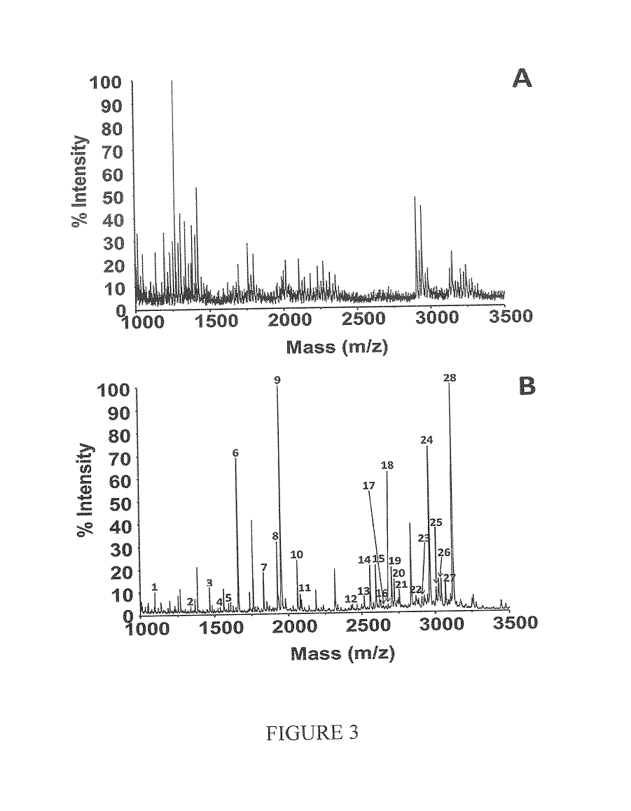

When the milk sample is analysed with the MALDI-MS (Matrix-assisted Laser Desorption/Ionization-Mass Spectrometry) directly, the mass spectrum given in the FIG. 3 is obtained (A). No signal of the phosphopeptides can be observed in this mass spectrum. After the process of phosphopeptide enrichment is realized using the syringe, when the sample is analysed only the mass spectrum wherein the signals of the phosphopeptides are located is obtained (B). This data indicates that phosphopeptides are separated from the other peptides and impurities in the medium successfully (FIG. 3).

It is possible to develop various embodiments of the inventive production method of sol-gel and use of sol-gel for enrichment of phosphopeptide, it cannot be limited to examples disclosed herein and it is essentially according to claims.

1. A production method of sol-gel (100) wherein the steps comprising of:
adding one or more of tantalum (V) alkoxide, tantalum (V) methoxide, tantalum (V) ethoxide, tantalum (V) propoxide, tantalum (V) isopropoxide and tantalum (V) butoxide into the mixture (101);
performing a mixing process during addition of material (102);
adding an acid solution such as HNO₃, HCl, H₂SO₄, CH₃COOH, CF₃COOH etc. during the mixing process (103);
decomposing the sol-gel with tantalum content through a centrifuge process (104).

2. A production method of sol-gel (100) according to claim 1, wherein before the addition of tantalum metal into the mixture (101), the solution comprising polyethylene glycol (PEG) (Mr-600-3000) in a ratio of 30-70% by volume is diluted with ethanol: water mixture (18:0.5-8:3.0, v/v) (1%, v/v).

3. A production method of sol-gel (100) according to claim 1, wherein while the mixing process continues, up to 0.1-0.5% of a 100% fuming HNO₃ solution is added into the synthesis medium (103).

4. A production method of sol-gel (100) according to claim 1, wherein the sol-gel with tantalum content obtained is washed and then taken into its final form after being dried.

5. A production method of sol-gel (100) according to claim 4, wherein the sol-gel with tantalum content is washed with solvents such as ethanol, water and methanol.

6. A production method of sol-gel (100) according to claim 4, wherein the drying process is carried out at temperature of 40° C. in a vacuum environment between 2-200 mbars.

7. A method (200) according to claim 1, wherein the method of phosphopeptide enrichment (200) which is realized using the sol-gel obtained comprises steps of:
inserting the sol-gel comprising the tantalum metal into the syringe (201);
adding some acid into the peptide mixture and adjusting PH (202);
drawing a certain amount of the solution generated into the syringe and releasing it (203);
carrying out the washing process (204); and
withdrawing the phosphopeptides from the syringe (205).

8. A method (200) according to claim 7, wherein after the sol-gel comprising the tantalum metal is inserted the into the syringe (201), firstly mixture of acetonitrile:water:trifluoroacetic acid (1:1:0.005-0.3, v/v/v) is added into the peptide mixture obtained as a result of enzymatic digestion and the PH is adjusted (202).

9. A method (200) according to claim 8, wherein in a preferred embodiment, 50 μL of the solution generated is drawn into the syringe and released (203).

10. A method (200) according to claim 9, wherein the process is repeated for 2-6 times.

11. A method (200) according to claim 7, wherein in order to remove impurities and peptides except phosphopeptides, the mixture of acetonitrile:water:trifluoroacetic acid (1:1:0.005-0.3, v/v/v) is drawn into the syringe and released.

12. A method (200) according to claim 11, wherein the process is repeated for 2-6 times.

13. A method (200) according to claim 7, wherein the solution of 10-100 mM ammonium bicarbonate comprising ammonia in the washing process is pulled through the syringe (204) lastly and the phosphopeptides are withdrawn from the syringe (205).
